(19) United States
(12) Patent Application Publication
(10) Pub. No.: US 2013/0095093 A1
(43) Pub. Date: Apr. 18, 2013

(54) METHODS AND COMPOSITIONS FOR TREATMENT OF IMMUNE DYSFUNCTION DISORDERS

(71) Applicants: Joy Campbell, Ames, IA (US); Ronald E. Strohbehn, Nevada, IA (US); Eric M. Weaver, Story City, IA (US); Barton S. Borg, Ames, IA (US); Louis E. Russell, Johnston, IA (US); Francisco Javier Polo Pozo, Barcelona (ES); John D. Arthington, Punta Gorda, FL (US); James D. Quigley, III, Johnston, IA (US)

(72) Inventors: Joy Campbell, Ames, IA (US); Ronald E. Strohbehn, Nevada, IA (US); Eric M. Weaver, Story City, IA (US); Barton S. Borg, Ames, IA (US); Louis E. Russell, Johnston, IA (US); Francisco Javier Polo Pozo, Barcelona (ES); John D. Arthington, Punta Gorda, FL (US); James D. Quigley, III, Johnston, IA (US)

(73) Assignee: APC, Inc., Ankeny, IA (US)

(21) Appl. No.: 13/463,127

(22) Filed: May 3, 2012

Related U.S. Application Data
(63) Continuation of application No. 10/470,981, filed on May 20, 2004, now abandoned.
Application No. 10/470,981 is a continuation-in-part of application No. 09/973,284, filed on Oct. 9, 2001, now abandoned.
Application No. 10/470,981 is a continuation of application No. PCT/US02/02752, filed on Jan. 29, 2002.
Application No. PCT/US02/02752 is a continuation of application No. 09/973,283, filed on Oct. 9, 2001, now abandoned.
(60) Provisional application No. 60/264,987, filed on Jan. 30, 2001.
(60) Provisional application No. 60/264,987, filed on Jan. 30, 2001.
(60) Provisional application No. 60/284,067, filed on Apr. 16, 2001.

(51) Int. Cl. A61K 39/395

(52) U.S. Cl. CPC A61K 39/395
USPC 424/130.1

(57) ABSTRACT

Methods and compositions are disclosed for modulating the immune system of animals. Applicant has identified that oral administration of immunoglubulin or plasma fractions purified from animal serum can modulate serum IgG and/or TNF-α levels for treatment of autoimmune disorders, potentiation of vaccination protocols, and improvement of overall health and weight gain in animals, including humans.

### CROSS-REFERENCE TO RELATED APPLICATIONS

This application is a continuation of and claims the benefit of priority under 35 U.S.C. §120 to U.S. patent application Ser. No. 10/470,981, entitled “METHODS AND COMPOSITIONS FOR TREATMENT OF IMMUNE DYSFUNCTION DISORDERS,” filed on May 20, 2004, which is a U.S. National Stage application under 35 U.S.C. 371 from International Application No. PCT/US02/02753 filed 29 Jan. 2002 and published in English as WO 02/078742 on 10 Oct. 2002, which is a continuation-in-part of U.S. patent application Ser. No. 09/973,284, filed 9 Oct. 2001 which claims priority under 35 U.S.C. 119(e) from U.S. Provisional Application Ser. No. 60/264,987, filed 30 Jan. 2001. U.S. patent application Ser. No. 10/470,981, entitled “METHODS AND COMPOSITIONS FOR TREATMENT OF IMMUNE DYSFUNCTION DISORDERS,” filed on May 20, 2004, is also a continuation of International Application No. PCT/US02/02752, filed 29 Jan. 2002 and published in English as WO 02/078741 on 10 Oct. 2002, which is a continuation-in-part of U.S. application Ser. No. 09/973,283, filed 9 Oct. 2001, which claims priority under 35 U.S.C. 119(e) from U.S. Provisional Application Ser. No. 60/264,987, filed 30 Jan. 2001 and U.S. Provisional Application Ser. No. 60/284,067, filed 16 Apr. 2001, all of which are incorporated herein by reference.

### BACKGROUND OF THE INVENTION

The primary source of nutrients for the body is blood, which is composed of highly functional proteins including immunoglobulin, albumin, fibrinogen and hemoglobin. Immunoglobulins are products of mature B cells (plasma cells) and there are five distinct immunoglobulins referred to as classes: M, D, E, A, and G. IgG is the main immunoglobulin class in blood. Intravenous administration of immunoglobulin products has long been used to attempt to regulate or enhance the immune system. Most evidence regarding the effects of intravenous IgG on the immune system suggests the constant fraction (Fc) portion of the molecule plays a regulatory function. The specific antigen binding properties of an individual IgG molecule are conferred by a three dimensional steric arrangement inherent in the amino acid sequences of the variable regions of two light and two heavy chains of the molecule. The constant region can be separated from the variable region if the intact molecule is cleaved by a proteolytic enzyme such as papain. Such treatment yields two fractions with antibody specificity (Fab fractions) and one relatively constant fraction (Fc). Numerous cells in the body have distinct membrane receptors for the Fc portion of an IgG molecule (Fcr). Although some Fcr receptors bind free IgG, most bind it more efficiently if an antigen is bound to the antibody molecule. Binding an antigen results in a configurational change in the Fc region that facilitates binding to the receptor. A complex interplay of signals provides balance and appropriateness to an immune response generated at any given time in response to an antigen. Antigen specific responses are initiated when specialized antigen presenting cells introduce antigen, forming a complex with the major histocompatibility complex molecules to the receptors of a specific helper inducer T-cells capable of recognizing that complex. IgG appears to be involved in the regulation of both allergic and autoimmune reactions. Intravenous immunoglobulin for immune manipulation has long been proposed but has achieved mixed results in treatment of disease states. A detailed review of the use of intravenous immunoglobulin as drug therapy for manipulating the immune system is described in Vol. 326, No. 2, pages 107-116, New England Journal of Medicine Dwyer, John M., the disclosure of which is hereby incorporated by reference.

There is a continuing effort and need in the art for improved compositions and methods for immune modulation of animals. Appropriate immunomodulation is essential to improve response to pathogens, vaccinations, for increasing weight gain and improving feed efficiency, improved health and for treatment of immune dysfunction disease states.

It is an object of the present invention to provide methods and pharmaceutical compositions for treating animals with immune dysfunction disease states.

It is yet another object of the invention to provide methods and compositions for immunomodulation of animals including humans for optimizing the response to antigens presented in vaccination protocols.

It is yet another object of the invention to increase weight gain, improve overall health and improve feed efficiency of animals by appropriately modulating the immune system of said animals.

It is yet another object of the invention to provide a novel pharmaceutical composition comprising purified plasma, components or derivatives thereof, which may be orally administered to create a serum IgG or TNF-α response.

These and other objects of the invention will become apparent from the detailed description of the invention which follows.

### SUMMARY OF THE INVENTION

According to the invention, applicants have identified purified and isolated plasma, components, and derivatives thereof, which are useful as a pharmaceutical composition for immune modulation of animals including humans. According to the invention, a plasma composition comprising immunoglobulin, when administered orally, regulates and lowers nonspecific immunity responses and induces a lowering and regulation of serum IgG levels and TNF-α levels relative to animals not orally fed immunoglobulin or plasma fractions. An orally administered plasma composition comprising immunoglobulin affects the animals overall immune status when exposed to an antigen, vaccination protocols, and for treatment of immune dysfunction disease states.

Applicants have unexpectedly shown that oral administration of plasma protein can induce a change in serum immunoglobulin and TNF-α as well as other non-specific immunity responses. This is unexpected as traditionally it was thought that plasma proteins such as immunoglobulins, must be introduced intravenously to affect concentration of circulating IgG, TNF-α or other components of nonspecific immunity. In contrast, applicants have demonstrated that oral globulin is able to impact circulating serum IgG, and TNF-α levels. Further this effect may be observed in as little as 14 days. This greatly simplifies the administration of immunomodulating compositions such as immunoglobulin as these compositions, according to the invention, can now be simply added to feedstuff or even water to modulate vaccination or to treat animals with immune dysfunction disease states.

Also according to the invention, applicants have demonstrated that modulation of serum IgG and TNF-α impacts the immune system response to stimulation as in vaccination protocols or to immune dysfunction disorders. Modulation of serum IgG, or TNF-α according to the invention allows the animals' immune system to more effectively respond to challenge by allowing a more significant up regulation response in the presence of a disease state or antigen presentation

Further this immune regulation impacts rate and efficiency of gain, as the bio-energetic cost associated with heightened immune function requires significant amounts of energy and nutrients which is diverted from such things as cellular growth and weight gain. Modulation of the immune system allows energy and nutrients to be used for other productive functions such as growth or lactation. See, Buttgerut et al., “Bioenergetics of Immune Functions: Fundamental and Therapeutic Aspects”, Immunology Today, April 2000, Vol. 21, No. 4, pp. 192-199.

Applicants have further identified that by oral consumption, the Fc region of the globulin composition is essential for communication and/or subsequent modulation of systemic serum IgG. This is unique, as this is the non-specific immune portion of the molecule which after oral consumption modulates systemic serum IgG without intravenous administration as previously noted (Dwyer, 1992). The antibody specific fractions produced less of a response without the Fc tertiary structure. Additionally, the globulin portion with intact confirmation gave a better reaction than the heavy and light chains when separated therefrom.

### BRIEF DESCRIPTION OF THE DRAWINGS

FIG. 1 is a graph depicting the effect of oral administration of plasma protein on antibody responses to a primary and secondary rotavirus vaccination.

FIG. 2 is a graph depicting the effect of oral administration of plasma proteins on antibody responses to a primary and secondary PRRS vaccination.

FIG. 3 is a graph depicting TNF-α in cultured macrophages: effect of LPS stimulation on TNF-α production. This graph depicts that suppressive effect of the oral administration of plasma proteins and fractions on TNF-α production.

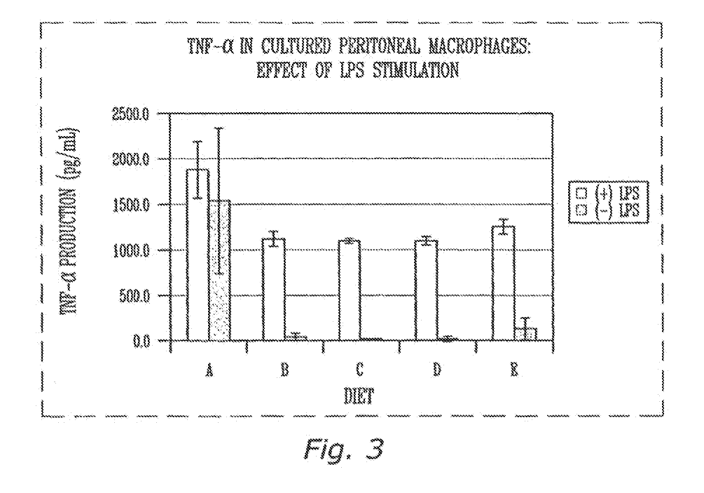

### DETAILED DESCRIPTION OF THE INVENTION

According to the invention, Applicant has provided herein a pharmaceutical composition comprising components purified and concentrated from animal plasma which are useful in practicing the methods of the invention. According to the invention gamma-globulin isolated from animal sources such as serum, plasma, egg, or milk is administered orally in conjunction with vaccination protocols, for treatment of various immune dysfunction disease states to modulate stimulation of the immune system. Quite surprisingly oral administration of this composition has been found to lower serum IgG and TNF-α levels relative to no administration of the pharmaceutical composition. Starting from a less stimulated state, the immune system is able to mount a more aggressive response upon challenge. Furthermore, disease states associated with elevated IgG or TNF-α levels are improved. As used herein with reference to the composition of the invention, the terms “plasma”, “globulin”, “gamma-globulin”, and “immunoglobulin” will all be used. These are all intended to describe a composition purified from animal sources including blood, egg, or milk which retains the Fc region of the immunoglobulin molecule. This also includes transgenic recombinant immunoglobulins purified from transgenic bacteria, plants or animals. This can be administered by spray-dried plasma, or globulin which has been further purified therefrom, or any other source of serum globulin which is available. One such source of purified globulin is NutraGammax™ or ImmunoLin™ available from Proliant Inc. Globulin may be purified according to any of a number of methods available in the art, including those described in Akita, E. M. and S. Nakai. 1993. Comparison of four purification methods for the production of immunoglobulins from eggs laid by hens immunized with an enterotoxigenic E. coli strain. Journal of Immunological Methods 160:207-214; Steinbuch, M. and R. Audran. 1969. The isolation of IgG from mammalian sera the aid of caprylic acid. Archives of Biochemistry and Biophysics 134:279-284; Lee, Y., T. Aishima, S. Nakai, and J. S. Sim. 1987. Optimization for selective fractionation of bovine blood plasma proteins using polyethylene glycol). Journal of Agricultural and Food Chemistry 35:958-962; Polson, A., G. M. Potgieter, J. F. Langier, G. E. F. Mears, and F. J. Toubert. 1964. Biochem. Biophys. Acta. 82:463-475.

Animal plasma from which immunoglobulin or other plasma fractions may be isolated include pig, bovine, ovine, poultry, equine, or goat plasma. Additionally, applicants have identified that cross species sources of the gamma globulins still provides the effects of the invention.

Concentrates of the product can be obtained by spray drying, lyophylization, or any other drying method, and the concentrates may be used in their liquid or frozen form. The active ingredient may also be microencapsulated, protecting and stabilizing from high temperature, oxidants, pH-like humidity, etc. The pharmaceutical compositions of the invention can be in tablets, capsules, ampoules for oral use, granulate powder, cream, both as a unique ingredient and associated with other excipients or active compounds, or even as a feed additive.

One method of achieving a gamma-globulin composition concentrate of the invention is as follows although the globulin may be delivered as a component of plasma.

The immunoglobulin concentrate is derived from animal blood. The source of the blood can be from any animal that has blood which includes plasma and immunoglobulins. For convenience, blood from beef, pork, and poultry processing plants is preferred. Anticoagulant is added to whole blood and then the blood is centrifuged to separate the plasma. Any anticoagulant may be used for this purpose, including sodium citrate and heparin. Persons skilled in the art can readily appreciate such anticoagulants. Calcium is then added to the plasma to promote clotting, the conversion of fibrinogen to fibrin; however other methods are acceptable. This mixture is then centrifuged to remove the fibrin portion.

Once the fibrin is removed from plasma resulting in serum, the serum can be used as a principal source of 1 g. Alternatively, one could also inactivate this portion of the clotting mechanism using various anticoagulants.

The defibrinated plasma is next treated with an amount of salt compound or polymer sufficient to precipitate the albumin or globulin fraction of the plasma. Examples of phosphate compounds which may be used for this purpose include all polyphosphates, including sodium hexametaphosphate and potassium polyphosphate. The globulin may also be isolated through the addition of polyethylene glycol or ammonium sulfate.

Following the addition of the phosphate compound, the pH of the plasma solution is lowered to stabilize the albumin precipitate. The pH should not be lowered below 3.5, as this will cause the proteins in the plasma to become damaged. Any type of acid can be used for this purpose, so long as it is compatible with the plasma solution. Persons skilled in the art can readily ascertain such acids. Examples of suitable acids are HCl, acetic acid, H₂SO₄, citric acid, and H₂PO₄. The acid is added in an amount sufficient to lower the pH of the plasma to the designated range. Generally, this amount will range from a ratio of about 1:4 to 1:2 acid to plasma. The plasma is then centrifuged to separate the globulin fraction from the albumin fraction.

The next step in the process is to raise the pH of the globulin fraction with a base until it is no longer corrosive to separation equipment. Acceptable bases for this purpose include NaOH, KOH, and other alkaline bases. Such bases are readily ascertainable by those skilled in the art. The pH of the globulin fraction is raised until it is within a non-corrosive range which will generally be between 5.0 and 9.0. The immunoglobulin fraction is then preferably microfiltered to remove any bacteria that may be present.

The final immunoglobulin concentrate can optionally be spray-dried into a powder. The powder allows for easier packaging and the product remains stable for a longer period of time than the raw globulin concentrate in liquid or frozen form. The immunoglobulin concentrate powder has been found to contain approximately 35-50% IgG.

In addition to administration with conventional carriers, active ingredients may be administered by a variety of specialized delivery drug techniques which are known to those of skill in the art. The following examples are given for illustrative purposes only and are in no way intended to limit the invention.

Those skilled in the medical arts will readily appreciate that the doses and schedules of the immunoglobulin will vary depending on the age, health, sex, size and weight of the patient rather than administration, etc. These parameters can be determined for each system by well-established procedures and analysis e.g., in phase I, II and III clinical trials.

For such administration the globulin concentrate can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose and the like.

In general, in addition to the active compounds, the pharmaceutical compositions of this invention may contain suitable excipients and auxiliaries which facilitate processing of the active compounds into preparations which can be used pharmaceutically. Oral dosage forms encompass tablets, dragees, and capsules.

The pharmaceutical preparations of the present invention are manufactured in a manner which is itself well known in the art. For example the pharmaceutical preparations may be made by means of conventional mixing, granulating, dragee-making, dissolving, lyophilizing processes. The processes to be used will depend ultimately on the physical properties of the active ingredient used.

Suitable excipients are, in particular, fillers such as sugars for example, lactose or sucrose, mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Dragee cores may be provided with suitable coatings which, if desired, may be resistant to gastric juices.

For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet of dragee coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition stabilizers may be added.

Oral doses of globulin or plasma protein according to the invention were found to modulate the primary and secondary immune response to rotavirus and PRRS vaccinations by helping to modulate IgG and the immune system.

Methods of the invention also include prevention and treatment of gastrointestinal diseases and infections, malabsorption syndrome, and intestine inflammation, and improving autoimmune states and reduction of systemic inflammatory reactions in humans and animals. The drug compositions, food and dietary preparations would be valid to improve the immune state in humans and animals, for diseases associated with elevated IgG, diseases associated with elevated TNF-α, diseases associated with immune regulatory dysfunction, for the support and treatment of malabsorption processes in humans and animals, and for treatment of clinical situations suffering from malnutrition in humans and animals. Among these malabsorption processes include syndrome of the small intestine, non-treatable diarrhea of autoimmune origin, lymphoma, postgastrectomy, steatorrhea, pancreas carcinoma, wide pancreatic resection, vascular mesentery failure, amyloidosis, scleroderma, eosinophilic enteritis. Clinical situations associated with malnutrition would include ulcerative colitis, Crohn's disease, cancerous cachexia due to chronic enteritis from chemo or radiotherapy treatment, and medical and infectious pathology comprising severe malabsorption such as AIDS, cystic fibrosis, enterocutaneous fistulae of low debit, and infantile renal failure.

The clinical uses of the composition would typically include disease states associated with immune dysfunction, particularly disease states associated with chronic immune stimulation. Examples of such diseases include but are not limited to myasthenia gravis, multiple sclerosis, lupus, polymyositis, Sjogren's syndrome, rheumatoid arthritis, insulin-dependent diabetes mellitus, bullous pemphigoid, thyroid-related eye disease, ureitis, Kawasaki's syndrome, chronic fatigue syndrome, asthma, Crohn's disease, graft-vs-host disease, human immunodeficiency virus, thrombocytopenia, neutropenia, and hemophilia.

Oral administration of IgG, TNF-α, or other plasma components to modulate circulating on specific immunity has tremendous advantages over parenteral administration. The most obvious are the risks associated with intravenous administration including: allergic reactions, the increased risk of disease transfer from human blood such as HIV or Hepatitis, the requirement for the same specie source, the cost of administration, and the benefits of oral IgG is greater neutralization of endotoxin and the “basal” stimulation of the immune system; the potential use of xenogeneic IgG. Applicants invention provides a non-invasive method of modulating the immune response. This can be used to treat autoimmune disorders (e.g. Rhesus reactions, Lupus, rheumatoid arthritis, etc.) and other conditions where immunomodulation, immunosuppression or immunoregulation is the desired outcome (organ transfers, chronic immunostimulatory disorders, etc.).

In another embodiment the invention can be used for oral immunotherapy (using antibodies) as an alternative to IVIG. But, prior to applicants' invention, one could not produce the massive amounts of antibodies required for sustained treatment because IVIG would require human IVIG. With oral administration of antibody, one can use a different specie source, without the threat of allergic reaction. This opens the door to milk, colostrum, serum, plasma, eggs, etc. from pigs, sheep, goats, cattle, etc. as the means of producing the relatively large amounts of immunoglobulin that would be required for sustained treatment.

The oral administration of antibody can:

1) Modulate the immunological response to exposure to a like/similar antigen. The data produced from the immunization of pigs with rotavirus or PRRS show that the oral administration of porcine immunoglobulin modifies the subsequent immune response to antigen administered intramuscularly. Communication occurs via the effects of IgG on the immune cells located in the GI tract (primarily the intestinal epithelium and lymphatic tissue). The plasma administered to the animals traditionally would contain antibody to both PRRS and rotavirus. Previous research has demonstrated that colostrum (maternal antibody) has this same effect when administered prior to gut closure. Applicant has demonstrated that antibody can modulate the immune response in an animal post gut-closure;

2) Serum IgG and TNF-α concentrations are lower with the oral administration of plasma proteins. This effect provides benefits to the prevention or treatment of much different conditions (e.g. Crohn's, IBD, IBS, sepsis, etc.) than the immunosuppressive effects of specific antibodies. This effect is not antibody specific. While not wishing to be bound by any theory it is postulated that plasma proteins can neutralize a significant amount of endotoxin in the lumen of the gut. In the newly weaned pig, that gut barrier function is compromised and will “leak” endotoxin. Endotoxin (LPS) is one of the most potent immunostimulatory compounds known. Thus as a post weaning aid, this invention can improve an animal's response to endotoxin by modulating the immune system preventing overstimulation. The route of feeding is important to the different effects. Parenteral feeding increases gut permeability and is known to substantially increase the likelihood of sepsis and endotoxemia when compared to enteral feeding. The oral supply of immunoglobulin improves gut barrier function and reduces the absorption of endotoxin. Diminished absorption of endotoxin would reduce the amount of endotoxin bound in plasma which would increase the plasma neutralizing capacity when compared to control animals.

The route of feeding is important to the different effects. Parenteral feeding increases gut permeability and is known to substantially increase the likelihood of sepsis and endotoxemia when compared to enteral feeding. The oral supply of immunoglobulin improves gut barrier function and reduces the absorption of endotoxin. Diminished absorption of endotoxin would reduce the amount of endotoxin bound in plasma which would increase the plasma neutralizing capacity when compared to control animals.

Applicants invention discloses immunomodulation, consistent with the observations of the effects of IVIG in the literature. Further, the immunomodulation effect of IgG was observed with different specie sources of IgG administered orally. This is very important to human medicine, particularly for autoimmune conditions (or cases where immunomodulation is desired).

### References:

Hardic, W. R. 1984. Oral immune globulin. U.S. Pat. No. 4,477,432. Filed Apr. 5, 1982.
Bier, M. Aug. 1, 2000. Oral immunotherapy of bacterial overgrowth. U.S. Pat. No. 6,096,310.
Bridger, J. C. and J. F. Brown. 1981. Development of immunity to porcine rotavirus in piglets protected from disease by bovine colostrum. Infection and Immunity 31:906.
Cunningham-Rundles, S. 1994. Malnutrition and gut immune function. Current Opinion in gastroenterology. 10:644-670.
Dwyer, J. M. 1992. Drug Therapy. Manipulating the Immune system with Immune Globulin. N.E.J.M. 326:107-116.
Eibl, M. M., H. M. Wolf, H. Furnkranz, and A Rosenkranz. 1988. Prevention of necrotizing enterocolitis in low-birth-weight infants by IgA-IgG feeding. N.E.J.M. 319:1-7.
Hammarstrom, L., A. Gardulf, V. Hammarstrom, A. Janson, K. Lindberg, and C. I. Edvard Smith. 1994. Systemic and topical immunoglobulin treatment in immunocompromised patients. Immunological Reviews 139:43-70.
Heneghan, J. B. 1984. Physiology of the alimentary tract. In: Coats, M. E., B. E. Gustafsson eds. The germ-free animal in biomedical research. London: Laboratory Animals Ltd. Pp. 169-191.
Henry, C. and N. Herne. 1968. J. Exp. Med. 128:133-152.
Karlsson, M. C. I., S. Wernersson, T. Diaz de stahl, S. Gustaysson, and B. Heyman. 1999. Efficient IgG-mediated suppression of primary antibody responses in Fclreceptor-deficient mice. Proc. Natl. Acad. Sci. 96:2244-2249.
Klobasa, F., J. E. Butler, and F. Habe, 1990. Maternal-neonatal immunoregulation: suppression of de novo synthesis of IgG and IgA, but not IgM, in neonatal pigs by bovine colostrum, is lost upon storage. Am. J. Vet. Res. 51:1407-1412.
McCracken, B. A., M. E. Spurlock, M. A. Roos, F. A. Zuckermann, and H. Rex Gaskins. Weaning anorexia may contribute to local inflammation in the piglet small intestine. J. Nutr. 129:613.
Mietens, C. and H. Keinhorst. 1979. Treatment of infantile E. coli gastroenteritis with specific bovine anti-E. coli milk immunoglobulins. Eur. J. Pediatr. 132:239-252.
O'Gormon, P., D. C. McMillan, and C. S. McArdle. 1998. Impact of weight loss, appetite, and the inflammatory response on quality of life in gastrointestinal cancer patients. Nutrition and Cancer 32(2):76-80.
Rowlands, B. J. and K. R. Gardiner. 1998. Nutritional modulation of gut inflammation. Proceedings of the Nutrition Society 57:395-401.
Sharma, R., U. Schumacher, V. Ronaasen, and M. Coates. 1995. Rat intestinal mucosal responses to a microbial flora and different diets. Gut 36:209-214.
Van der Poll, T., M. Levi, C. C. Braxton, S. M. Coyle, M. Roth, J. W. Ten Cate, and S. F. Lowry. 1998. Parenteral nutrition facilitates activation of coagulation but not fibrinolysis during human endotoxemia. J. Infect. Dis. 177:793-795.
Wolf, H. M. and M. M. Eibl. 1994. The anti-inflammatory effect of an oral immunoglobulin (IgA-IgG) preparation and its possible relevance for the prevention of necrotizing enterocolitis. Acta Pediatr. Suppl. 396:37-40.
Skarnes, R. C. 1985, In vivo distribution and detoxification of endotoxins. In: Proctor, R. A. (ed): Handbook of Endotoxin, Vol. 3, Pp. 56-81.
Zhang, G. H., L. Baek, T. Bertelsen and C. Kock. 1995. Quantification of the endotoxin-neutralizing capacity of serum and plasma. APMIS 103:721-730.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

### EXAMPLE 1

### Preferred Manufacturing Method For Globulin Concentrate

The following illustrates a preferred method of manufacturing the globulin concentrate of the present invention.

### EXAMPLE 2

### Necessity of Intact Globulin

Previous research demonstrates that oral plasma consumption improves weanling pig performance (Coffey and Cromwell, 1995). Data indicates that the high molecular weight fraction present in plasma influences the performance of the pig (Cain, 1995; Owen et al, 1995; Pierce et al., 1995, 1996; Weaver et al., 1995). The high molecular weight fraction is composed primarily of IgG protein. Immunoglobulin G protein is approximately 150,000 MW compound consisting of two 50,000 MW polypeptide chains designated as heavy chains and two 25,000 MW chains, designated as light chains (Kuby, 1997). An approach to hydrolysis of intact IgG has been demonstrated in the lab with the enzyme pepsin. A brief digestion with pepsin enzyme will produce a 100,000 MW fragment composed of two Fab-like fragments (Fab=antigen-binding). The Fc fragment of the intact molecule is not recovered as it is digested into multiple fragments (Kuby, 1997). A second type of processing of the globulin-rich concentrate is by disulfide bond reduction with subsequent blocking to prevent reformation of disulfide bonds. The resulting reduced sections from the globulin molecule are free intact heavy and light chains.

In the first example the objective was to quantify the impact by oral consumption of different plasma fractions and pepsin hydrolyzed plasma globulin on average daily gain, average daily feed intake, intestinal morphology, blood parameters, and intestinal enzyme activity in weanling pigs.

### Materials and Methods

Animals and Diets. Sixty-four individually penned pigs averaging 6.85 kg body weight and 21 d of age were allotted to four dietary treatments in a randomized complete block design. Two rooms of 32 pens each were used. The nursery rooms previously contained animals from the same herd of origin and were not cleaned prior to placement of the test animals to stimulate a challenging environment. Pigs were given ad libitum access to water and feed.

Dietary treatments are represented in Table 1 consisting of: 1) control; 2) 6% spray-dried plasma; 3) 3.6% spray-dried globulin; and 4) 3.6% spray-dried pepsin digested globulin. Diets are corn-soybean meal-dried whey based replacing menhaden fishmeal with plasma on an equal protein basis. Plasma fractions were included, relative to plasma, on an equal plasma fraction basis. Diets contained 1.60% lysine were formulated to an ideal amino acid profile (Chung and Baker, 1992). Diets were pelleted at 130° F. or less and were fed from d 0-14 post-weaning

Collection of Data. Individual pig weights were collected on d 0, 2, 4, 6, 8, 10, 12, and 14 post-weaning. Feed intake and diarrhea score were collected daily from d 0 to 14 post-weaning Blood was collected d 0, 7, and 14 post-weaning The blood was centrifuged and serum was frozen for subsequent analysis. Upon completion of the study (d 14), six randomly selected pigs/treatment were sacrificed to obtain samples for measurement of villous height, crypt depth, intestinal enzyme activity, and organ weights (intestine, liver, lung, heart, spleen, thymus, kidney, stomach, and pancreas). Immediately after euthanasia, the body cavity was opened and the ileal-cecal juncture was located. The small intestine was removed and dissected free of mesenteric attachment. One meter cranial to the ileal-cecal juncture, 10 cm of intestine (ileum) was removed and fixed in phosphate-buffered formalin for subsequent histology measurements. From the midsection of the duodenum, the mucosa was scraped, weighed, and frozen for subsequent enzymatic analysis.

Histology. The jejunal samples were paraffin embedded and stained with hematoxylin and eosin (H&E) and were analyzed using light microscopy to measure crypt depth and villous height. Five sites were measured for crypt depth and villous height on each pig.

Enzyme analysis. Lactase and maltase activity were measured on the mucosal scrapings according to Dahlqvist, 1964.

Serum analysis. Total protein and albumin were analyzed according to ROCHE Diagnostic kits for a COBAS MIRA system. Serum IgG was analyzed according to Etzel et al. (1997).

Statistical Analysis. Data were analyzed as a randomized complete block design. Pigs were individually housed and the pen was the experimental unit. Analysis of variance was performed using the GLM procedures of SAS (SAS/STAT Version 6.11 SAS Institute, Cary, N.C.). Model sum of squares consisted of block and treatment, using initial weight as a covariate. Least squares means for treatments are reported.

### Results

Average daily gain (ADG) and average daily feed intake (ADFI) are presented in Table 2. No differences were noted for ADG or ADFI from d 0-6. From d 0-14, plasma and globulin improved (P<0.05) ADG and ADFI compared to the control, while the pepsin digested globulin treatment was intermediate. Organ weights were recorded and expressed as g/kg of body weight (Table 3). No differences were noted in heart, kidney, liver, lung, small intestine, stomach, thymus, or spleen; however, pancreas weight was increased (P<0.05) due to inclusion of globulin and pepsin digested globulin compared to the control. The plasma treatment was intermediate. Blood parameters are presented in Table 4. Compared to the control, serum IgG of globulin fed pigs (d 14) was lower (P<0.08), while that of the plasma and pepsin digested globulin treatments were intermediate. No differences (P>0.10) were noted in total protein. Serum albumin was increased (P<0.08) on d 14 with the globulin and plasma treatment compared to the control, while that of the pepsin digested globulin group was intermediate. Enzyme activity, intestinal morphology, and fecal score are presented in Table 5. No differences (P>0.10) were noted in villous height and crypt depth. Duodenal lactase and maltase activity was increased (P<0.07) due to consumption of pepsin digested globulin compared to the control diet, while the other dietary treatments were intermediate. The fecal score was reduced (P<0.07; representing a firmer stool) due to the addition of pepsin digested globulin compared to the control while the fecal score of and plasma while globulin was intermediate.

### Tables

TABLE 1
Composition of experimental diets (as fed, %).^{a}

| | | | | Pepsin |
|---|---|---|---|---|
| | | | | Digested |
| Ingredients | Control | Plasma | Globulin | Globulin |
| Corn | 42.932 | 43.012 | 42.962 | 42.957 |
| 47% SBM | 23.000 | 23.000 | 23.000 | 23.000 |
| Dried Whey | 17.000 | 17.000 | 17.000 | 17.000 |
| Menhaden | 8.500 | | 3.400 | 3.400 |
| Fishmeal | | | | |
| Plasma | | 6.000 | | |
| Globulin | | | 3.600 | |
| Pepsin Digested | | | | 3.600 |
| Globulin | | | | |
| Soy Oil | 4.300 | 5.100 | 4.800 | 4.800 |
| Lactose | 2.118 | 2.118 | 2.118 | 2.118 |
| 18.5% Dical | 0.400 | 1.700 | 1.150 | 1.150 |
| Limestone | 0.070 | 0.435 | 0.290 | 0.290 |
| Zinc Oxide | 0.400 | 0.400 | 0.400 | 0.400 |
| Mecadox | 0.250 | 0.250 | 0.250 | 0.250 |
| Salt | 0.250 | 0.250 | 0.250 | 0.250 |
| Premix | 0.400 | 0.400 | 0.400 | 0.400 |
| L-Lysine HCL | 0.250 | 0.195 | 0.290 | 0.290 |
| L-Threonine | 0.090 | | | |
| DL-Methionine | 0.040 | 0.140 | 0.090 | 0.095 |

^{a}Diets were formulated to contain 1.60% lysine, 0.48% methionine, 14% lactose, 0.8% calcium, and 0.7% phosphorus and fed from d 0 to 14 post-weaning.

TABLE 2
Effect of spray-dried plasma and plasma fractions
on average daily gain and feed intake (kg/d).¹

| | | | | Pepsin | |
|---|---|---|---|---|---|
| | | | | Digested | |
| Treatment | Control | Plasma | Globulin | Globulin | SEM |
| ADG, kg/d | | | | | |
| D 0-6 | 0.037 | 0.094 | 0.080 | 0.073 | 0.029 |
| D 0-14 | 0.169^{a} | 0.242^{b} | 0.234^{b} | 0.222^{ab} | 0.025 |
| ADFI, kg/d | | | | | |
| D 0-6 | 0.104 | 0.134 | 0.132 | 0.128 | 0.018 |
| D 0-14 | 0.213^{a} | 0.276^{b} | 0.278^{b} | 0.254^{ab} | 0.021 |

¹Values are least squares means with 16 pigs/treatment.
^{ab}Means within a row without common superscript letters are different (P < 0.10).

TABLE 3
Effect of spray-dried plasma and plasma fractions
on organ weights (g/kg body weight)¹

| | | | | Pepsin | |
|---|---|---|---|---|---|
| Organ Weights, | | | | Digested | |
| g/kg BW | Control | Plasma | Globulin | Globulin | SEM |
| Intestine | 44.21 | 50.65 | 50.34 | 44.71 | 3.43 |
| Liver | 32.34 | 31.20 | 30.23 | 32.27 | 1.42 |
| Spleen | 1.74 | 1.83 | 1.81 | 2.06 | 0.16 |
| Thymus | 1.45 | 1.39 | 1.32 | 1.36 | 0.20 |
| Heart | 4.93 | 4.89 | 4.94 | 4.73 | 0.22 |
| Lung | 11.26 | 11.28 | 12.14 | 11.95 | 1.03 |
| Stomach | 6.96 | 7.06 | 6.61 | 6.84 | 0.32 |
| Kidney | 4.76 | 5.75 | 5.66 | 5.45 | 0.47 |
| Pancreas | 1.93^{a} | 2.20^{ab} | 2.42^{b} | 2.34^{b} | 0.11 |

¹Values are least squares means of 6 pigs/treatment.
^{ab}Means within a row without common superscript letters are different (P < 0.05).

TABLE 4
Effect of spray-dried plasma and plasma
fractions on blood parameters.^{1,2}

| | | | | Pepsin | |
|---|---|---|---|---|---|
| | | | | Digest | |
| | Control | Plasma | Globulin | Globulin | SEM |
| IgG, mg/mL | | | | | |
| D0 | 4.84^{a} | 5.70^{b} | 4.83^{a} | 5.05^{ab} | 0.34 |
| D7 | 4.98 | 4.71 | 4.66 | 4.96 | 0.17 |
| D14 | 4.88^{b} | 4.43^{ab} | 4.30^{a} | 4.54^{ab} | 0.24 |
| Total Protein, g/dL | | | | | |
| D0 | 4.55 | 4.59 | 4.54 | 4.65 | 0.07 |
| D7 | 4.39 | 4.37 | 4.35 | 4.47 | 0.08 |
| D14 | 4.22 | 4.30 | 4.29 | 4.20 | 0.07 |
| Albumin, g/dL | | | | | |
| D0 | 3.03 | 3.02 | 3.11 | 3.09 | 0.06 |
| D7 | 2.98 | 3.03 | 3.02 | 3.01 | 0.06 |
| D14 | 2.61^{a} | 2.78^{b} | 2.80^{b} | 2.71^{ab} | 0.07 |

¹Values are least squares means of 16 pigs/treatment.
²Day 0 used as a covariate for analysis on D7 and D14.
^{ab}Means within a row without common superscript letters are different (P < 0.08).

TABLE 5
Effect of spray-dried plasma and plasma fractions on
enzyme activities, intestinal morphology, and fecal score.¹

| | | | | Pepsin | |
|---|---|---|---|---|---|
| | | | | Digested | |
| | Control | Plasma | Globulin | Globulin | SEM |
| Maltase, umol/ | 7.97^{a} | 11.08^{ab} | 10.93^{ab} | 13.30^{b} | 1.93 |
| mg prot/hr | | | | | |
| Lactase, umol/ | 1.14^{a} | 1.57^{ab} | 1.55^{ab} | 2.15^{b} | 0.31 |
| mg prot/hr | | | | | |
| Villous Height, | 378.7 | 370.7 | 374.0 | 387.7 | 34.4 |
| micron | | | | | |
| Crypt Depth, | 206.3 | 191.0 | 195.0 | 192.7 | 9.3 |
| micron | | | | | |
| Fecal Score | 5.12^{b} | 5.06^{b} | 4.19^{ab} | 2.88^{a} | 0.65 |

¹Values are least squares means of 6 pigs/treatment
^{ab}Means within a row without common superscript letters are different (P < 0.07)

### EXAMPLE 3

### Quantity and Impact of Dietary Inclusion of Variable Plasma Fractions

In the second experiment the objective was to quantify the impact of dietary inclusion of different plasma fractions and the effect of separating the heavy and light chains of the IgG on average daily gain, average daily feed intake, organ weights, and blood parameters of weanling pigs.

### Materials and Methods

Animals and Diets. Ninety-six individually penned pigs averaging 5.89 kg body weight and 21 d of age were allotted to four dietary treatments in a randomized complete block design. The animals were blocked by time between 3 unsanitized nursery rooms. Pigs were given ad libitum access to water and feed.

Dietary treatments (Table 6) consisted of: 1) control; 2) 10% spray-dried plasma; 3) 6% sp1·ay-dried globulin; and 4) 6% globulin-rich material treated to reduce the disulfide bonds of the IgG molecule (H+L). Diets were corn-soybean meal-dried whey based replacing soybean meal with plasma on an equal lysine basis. The plasma fractions were added relative to plasma on an equal plasma fraction basis. Diets contained 1.60% lysine and were formulated to an ideal amino acid profile (Chung and Baker, 1992). Diets were meal form and fed from d 0-14 post-weaning.

Collection of Data. Individual pig weights were collected on d 0, 2, 4, 6, 8, 10, 12, and 14 post-weaning. Feed intake and diarrhea score were collected daily from d 0 to 14 post-weaning Blood was collected on d 0, 7, and 14 post-weaning. The blood was centrifuged and serum samples were frozen for subsequent analysis. Upon completion of the study (d 14), nine pigs/treatment were sacrificed to obtain organ weights (intestine, heart, liver, spleen, thymus, lung, kidney, stomach, and pancreas).

Serum Analysis. Total protein, albumin, and urea nitrogen were analyzed according to ROCHE Diagnostic kits for a COBAS MIRA system. Serum IgG was analyzed according to Etzel et al. (1997).

Statistical Analysis. Data were analyzed as a randomized complete block design using the GLM procedures of SAS (SAS/STAT Version 6.11 SAS Institute, Cary N.C.). Pigs were individually housed and the pen was the experimental unit. Model sum of squares consisted of block and treatment, using initial weight as a covariate. Least squares means for treatments are reported.

### Results

From d 0-6 (Table 7), plasma increased (P<0.10) ADFI compared to control and H+L, while the globulin was intermediate. From d 7-14, plasma increased (P<0.10) ADFI compared to control and H+L treatments. Average daily feed intake of globulin fed pigs was increased compared to the control. From d 0-14, plasma and globulin increased (P<0.10) ADFI compared to the control and H+L dietary treatments. Average daily gain is presented in Table 8. Average daily gain was similar to ADFI ford 0-6. From d 7-14 and 0-14, plasma and globulin increased (P<0.10) ADG compared to the control, while H+L was intermediate. Blood parameters are presented in Table 9. Serum IgG and urea nitrogen (d 14) were lower (P<0.05) by the dietary inclusion of plasma and globulin compared to the control. The effect of H+L was intermediate. Dietary treatment had no effect on serum protein. Serum albumin (d 7) was decreased (P<0.05) due to inclusion of plasma compared to the other dietary treatments. No differences were noted in fecal score. Intestinal length and organ weights are presented in Table 10. No differences were noted in organ weights or intestinal length due to dietary treatment.

TABLE 6
Composition of experimental diets (as fed. %)¹

| Ingredients | Control | Plasma | Globulin | H + L |
|---|---|---|---|---|
| Corn | 37.937 | 44.96 | 40.006 | 40.034 |
| 47% Soybean Meal | 18 | 18 | 18 | 18 |
| Dried Whey | 14 | 14 | 14 | 14 |
| Lactose | 6.253 | 6.253 | 6.253 | 6.253 |
| Plasma | | 10 | | |
| Globulin | | | 6 | |
| H + L | | | | 6 |
| Soy Protein | 17.31 | | 9.07 | 9.07 |
| Concentrate | | | | |
| Soy Oil | 3.219 | 3.047 | 3.187 | 3.186 |
| 18.5% Dical | 1.79 | 1.493 | 2.133 | 2.146 |
| Limestone | 0.562 | 0.354 | 0.46 | 0.42 |
| Premix | 0.55 | 0.55 | 0.55 | 0.55 |
| Salt | 0.15 | 0.15 | 0.15 | 0.15 |
| DL-Methionine | 0.083 | 0.152 | 0.092 | 0.096 |
| L-Lysine HCL | 0.146 | 0.041 | 0.099 | 0.095 |

¹Diets were formulated to contain 1.60% lysine, 0.48% methionine, 16% lactose, 0.9% calcium, and 0.8% phosphorus and fed from d 0 to 14 post-weaning.

TABLE 7
Effect of spray-dried plasma and plasma fractions
on average daily feed intake (g/d).¹

| | Control | Plasma | Globulin | H + L | SEM |
|---|---|---|---|---|---|
| ADFI, g/d | | | | | |
| D 0-6 | 102.82^{a} | 152.43^{b} | 128.53^{ab} | 114.50^{a} | 13.44 |
| D 7-14 | 280.74^{a} | 413.57^{c} | 379.21^{bc} | 319.06^{ab} | 29.07 |
| D 0-14 | 193.94^{a} | 284.83^{b} | 258.55^{b} | 216.83^{ab} | 16.69 |

¹Values are least squares means of 24 pigs/treatment.
^{abc}Means within a row without common superscript letters are different (P < 0.10).

TABLE 8
Effect of spray-dried plasma and plasma
fractions on average daily gain (g/d).¹

| | Control | Plasma | Globulin | H + L | SEM |
|---|---|---|---|---|---|
| ADG, g/d | | | | | |
| D 0-6 | −41.05^{a} | 27.23^{b} | −1.23^{ab} | -21.86^{a} | 20.26 |
| D 7-14 | 199.38^{a} | 282.46^{b} | 302.22^{b} | 255.12^{ab} | 26.40 |
| D 0-14 | 96.34^{a} | 173.07^{b} | 172.17^{b} | 136.42^{ab} | 20.56 |

¹Values are least squares means of 24 pigs/treatment.
^{abc}Means within a row without common superscript letters are different (P < 0.10).

TABLE 9
Effects of spray-dried 2Iasma fractions on blood parameters.^{1,2}

| | Control | Plasma | Globulin | H + L | SEM |
|---|---|---|---|---|---|
| IgG, g/dL | | | | | |
| D 0 | 0.674 | 0.664 | 0.584 | 0.661 | 0.037 |
| D 7 | 0.668 | 0.643 | 0.624 | 0.673 | 0.021 |
| D 14 | 0.631^{b} | 0.555^{a} | 0.545^{a} | 0.596^{ab} | 0.022 |
| Urea N. mg/dL | | | | | |
| D 0 | 8.53 | 9.78 | 9.94 | 9.87 | 0.68 |
| D 7 | 17.55^{b} | 14.65^{a} | 16.48^{ab} | 17.56^{b} | 1.01 |
| D 14 | 17.57^{c} | 10.48^{a} | 14.73^{b} | 15.56^{bc} | 0.87 |
| Total Protein, g/dL | | | | | |
| D 0 | 4.58 | 4.46 | 4.56 | 4.56 | 0.076 |
| D 7 | 4.69 | 4.60 | 4.53 | 4.74 | 0.106 |
| D 14 | 4.55 | 4.49 | 4.59 | 4.49 | 0.080 |
| Albumin, g/dL | | | | | |
| D 0 | 2.69 | 2.64 | 2.75 | 2.69 | 0.069 |
| D 7 | 2.92^{b} | 2.79^{a} | 2.92^{b} | 2.94^{b} | 0.045 |
| D 14 | 2.83 | 2.76 | 2.86 | 2.80 | 0.060 |

¹Values are least squares means of 24 pigs/treatment.
²Day 0 used as a covariate for analysis on D7 and D14.
^{abc}Means within a row without common superscript letters are different (P < 0.05).

TABLE 10
Effect of spray-dried plasma and plasma fractions on intestinal
length (inches) and organ weights (g/kg body weight)¹

| | Control | Plasma | Globulin | H + L | SEM |
|---|---|---|---|---|---|
| Int. length, inch | 358.67 | 368.33 | 359.33 | 358.56 | 13.05 |
| Organ weight, g/kg BW | | | | | |
| Intestine | 41.48 | 41.79 | 42.82 | 41.04 | 2.16 |
| Liver | 29.61 | 32.61 | 32.29 | 31.09 | 1.10 |
| Spleen | 2.05 | 2.32 | 2.44 | 2.17 | 0.22 |
| Thymus | 1.15 | 1.45 | 1.15 | 1.15 | 0.14 |
| Heart | 6.12 | 6.14 | 5.77 | 5.80 | 0.22 |
| Lung | 12.24 | 12.33 | 13.65 | 11.63 | 0.74 |
| Stomach | 9.26 | 9.14 | 10.08 | 10.08 | 0.58 |
| Kidney | 6.18 | 6.57 | 6.10 | 6.30 | 0.21 |
| Pancreas | 2.70 | 2.61 | 2.54 | 2.70 | 0.11 |

¹Values are least squares means of 9 pigs/treatment.

### Discussion

Consistent with published research (Coffey and Cromwell, 1995) these data indicate that when included in the diet plasma and globulin increase performance (ADG, ADFI) compared to the control. The pepsin digested globulin and H+L fraction resulted in an intermediate improvement in performance. Enzyme activity (lactase and maltase) were increased and fecal score was improved with the addition of all plasma fractions (plasma, globulin, pepsin digested globulin, H&L) compared to the control.

Serum IgG concentration and BUN were lower after consumption of plasma or globulin treatments compared to the control, pepsin digested globulin or H&L. The ability of oral plasma or globulin administration to elicit a systemic response as demonstrated by lower serum IgG compared to the control was unexpected.

The noted differences between plasma and globulin fractions compared to the pepsin digested globulin or H+L is that the tertiary structure of the Fc region is intact in the plasma and globulin fractions only. The pepsin digested globulin has the Fc region digested, while in the H+L fraction, the Fc region remains intact but without tertiary confirmation. The Fab region is still intact in the pepsin digested globulin. The variable region is still able to bind antigen in the H+L preparation (APC, unpublished data). Thus, the results indicate the antibody-antigen interaction (Fab region) is important for local effects (reduced fecal score, increased lactase and maltase activity), while the intact Fab and Fc region of plasma and globulin fractions is important to modulate the systemic serum IgG response.

### EXAMPLE 4

### Effect of Oral Doses of Plasma Protein on Active Immune Responses to Primary and Secondary Rotavirus and PRRS Vaccinations in Baby Pigs

### Overview

To examine the influence of supplemental plasma protein on active immune responses following primary and secondary rotavirus and PRRS vaccinations.

### Methods

Ten sows induced to farrow at a common time were utilized. Treatments were assigned randomly within each litter. Treatment delivery occurred twice weekly (3 or 4 day intervals) via a stomach tube applicator. A series of 7 applications occurred prior to the final vaccination and weaning Treatments consisted of: control (10 mL saline) and plasma IgG (0.5 g delivered in a final volume of 8 mL). All pigs received a primary vaccination (orally=rotavirus; injection=PRRS) 10 days prior to weaning A secondary vaccination was given at the time of weaning via intramuscular injection. Blood samples were collected prior to the primary vaccination (10 d prior to weaning), prior to the secondary vaccination (at weaning), and on 3 day intervals until 12 days post-weaning

### Results

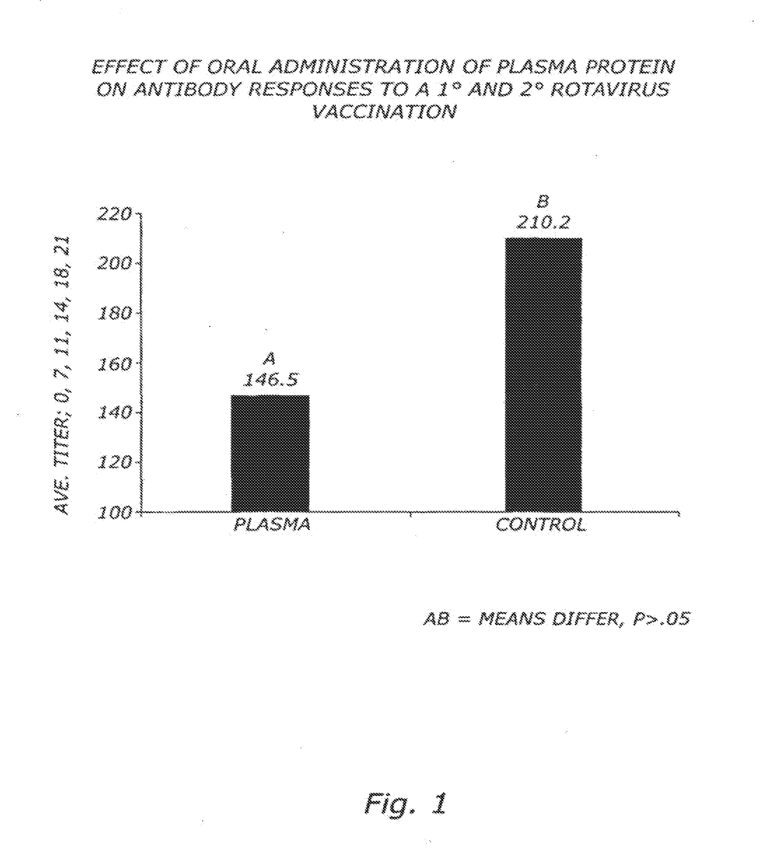

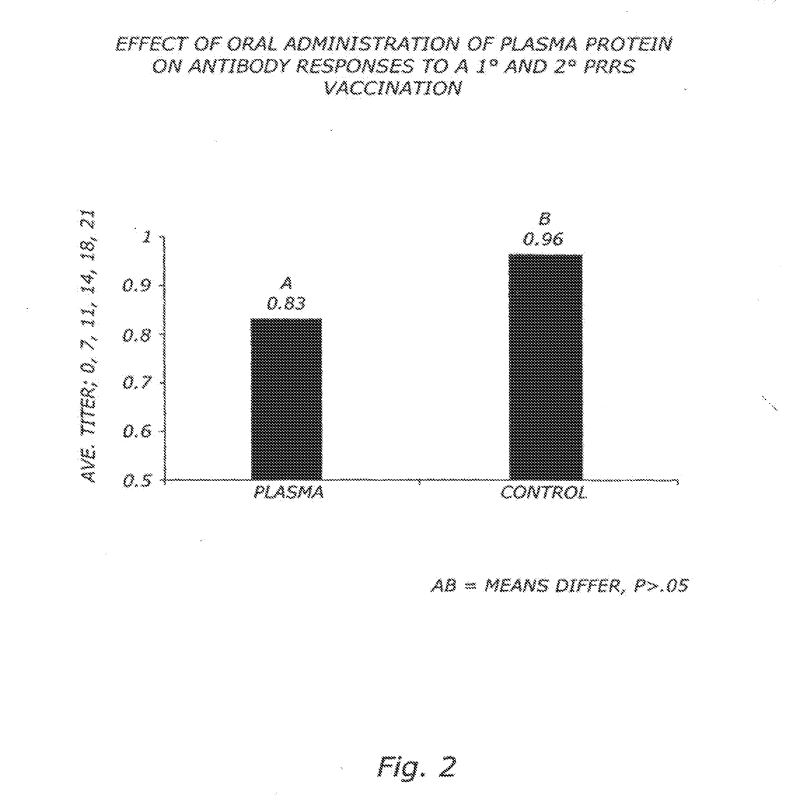

Pigs dosed with plasma protein experienced significant (P<0.05) decreases in specific antibody titers following booster vaccination. This response was seen for both rotavirus (FIG. 1) and PRRS (FIG. 2) antibody titers.

### Discussion

These data provide an excellent indication of the effect of oral plasma protein in the young pig. Immune activation acts as a large energy and nutrient sink. When the immune system is activated energy and nutrients are funneled into the production of immune products (immunoglobulin, cytokines, acute phase proteins, etc.) and away from growth. Oral plasma may modulate the immune system, thereby allowing energy and nutrients to be redirected to other productive functions such as growth.

### EXAMPLE 5

### The Effects of Orally-administered Plasma on Immunological Functions

The immunological response to plasma protein administration has not been elucidated. However, some of the individual components from colostrum or milk have been found to have immuno-modulatory effects. IgA and sIgA have anti-inflammatory functions in neonates¹⁻³. Eibl found that the oral administration of human immunoglobulin reduces circulating TNF-α production by isolated macrophages and also reduces immunoglobulin concentrations in young children affected by necrotizing enterocolitis¹. Schriffrin found that colostrum was effective in the modulation of experimental colitis⁴. In an uncontrolled study, Schriffrin and his colleagues found that the dietary supplementation of a TGF-β2-rich casein fraction was useful in the modulation of inflammation in Crohn's disease⁵. The mode of action has not been elucidated but TGF-β2 has been found to inhibit interferon-γ induced MHC Class II receptor expression in neonates⁶. MHC class II receptor expression is known to be upregulated in newly weaned animals⁷. Other peptides found in milk, colostrum, and plasma could also have anti-inflammatory effects. TGF-β1 has been shown to improve survival of mice challenged with salmonella.

TNF-α is a central cytokine in inflammatory processes and has negative effects on appetite and protein utilization^{8,9}. And, it is well-known that the production of TNF-α is stimulated with exposure of phagocytes to endotoxin. Plasma proteins contain immunoglobulin, endotoxin-binding proteins, mannan-binding lectins, and TGF-β. All of these proteins could play a role in reducing the exposure of the immune system to lumen-derived endotoxin and therefore alter the activation of the immune system. In addition, immunomodulatory effects of TGF-β could alter the responsiveness of the immune system to endotoxin.

The objective of this experiment was to study the immunomodulatory effects of plasma protein administration in animals beyond the postweaning period through measurement of: (a) respiratory burst in peripheral blood monocytes, (b) respiratory burst in peritoneal macrophages, (c) phagocytosis in peritoneal macrophages, and (d) TNF-α production of peritoneal macrophages in the presence and absence of Lipopolysaccharide.

### 2.0 Study Design

### 2.1 Animals

60 Balb/c White female mice were received from Charles River Laboratories. Upon receipt, the animals were housed four per cage. At start of dosing the body weight range was 15-19 g. Three cages were assigned to a test diet, for a total of 12 animals per diet. The dosing had to be staggered on three successive days to accommodate the processing required at necropsy. So that on day 1 after arrival dosing was initiated on the animals in cage 1 from each treatment/control group, on day 2 the dosing was initiated in all the second cages, and on day 3 the third cages from all groups were dosed. Necropsy was similarly staggered so that the animals were dosed for a total of 7 days. All cages were labeled with the animal numbers and designated diet. The animal room was maintained between 66 and 82 θF. The lighting was on a 12 hours on-12 hours off cycle.

### 2.2 Peritoneal Lavage, Bleeding and Blood Sample Processing

Cells were harvested from each animal by peritoneal lavage. After termination, the abdominal muscles were drawn away from the abdominal organs and 9 ml of sterile PBS was injected into the peritoneal cavity. The abdomen was massaged and 6-8 ml of lavage fluid was recovered. The four mice housed together were pooled to form one sample. The samples were kept on ice prior to processing. The cells were centrifuged and the pellet was re-suspended in 1 ml of Dulbeccco's Modified Eagle's Medium (DMEM) with Fetal Bovine Serum and Penicillin/Streptomycin. The cell numbers were determined using a Coulter Counter Z1.

After collecting the lavage cells, the abdominal cavity was opened and blood was collected from the renal artery and transferred to a 3 ml vacutainer tube containing EDTA. Once again four mice were pooled to form one sample. The blood samples were diluted in PBS for a total volume of 8 ml. This mixture was then layered on top of 3 ml of Histopaque®-1077. The samples were centrifuged and the opaque interface containing the mononuclear cells was removed with a pasteur pipette. After a total of three washes in PBS the pellet was re-suspended in 0.5 ml PBS. The cell numbers were determined using a Coulter Counter Z1.

### 2.3 Respiratory Burst

After the cell counts were determined, both the monocyte and peritoneal samples were adjusted to a concentration of 1×10⁶ cells per ml. All samples were assayed in triplicate. One hundred (100) ul of each cell suspension (1×10⁵ cells/well) was added to a 96-well tissue culture plate. 2,7-Dicholorofluorescein diacetate (Molecular Probes) was added to each well and the plate was incubated at 37° C. to allow uptake of the substrate by the cells. Following incubation, Phorbol Myristate Acetate (PMA) (Sigma) was added to triplicate wells of at a concentration of 10 ng/well in order to stimulate oxygen radical production. The plate was incubated at 37° C. After the 1-hour incubation, 200 ul of each 2,7-dicholorofluorescein standard (Polysciences) was added to the plate. The increase in fluorescent product was then measured using the Cytofluor 4000 (PerSeptive Biosystems) fluorescence microplate reader (Wavelengths: excitation—485, emission—530). The data was exported from the Cytofluor program into Excel. From Excel the plate layout was copied then pasted into a Softmax Pro file (Molecular Devices), where the results were determined automatically by interpolation of the standard curve.

### 2.4 Phagocytosis

One hundred (100) ul of each cell suspension was added to five wells on a 96-well tissue culture plate, at a concentration of 1×10⁶ cells per ml (1×10⁵ cells/well). 50 ul of medium (DMEM) was added to each well, making the final volume 150 ul. Five wells containing only DMEM were used as plate blanks Each samples or blank was run in a set of five (5) replicates. The cells were incubated at 37° C. and then examined under a microscope.

During the incubation period, the E. coli K-12 bioparticle suspension in HBSS (Molecular Probes) was prepared. The mixture was vortexed and sonicated. After the one-hour incubation period, the plates were centrifuged, and the supernate was aspirated by vacuum aspiration. 100 ul of the E. coli/HBSS mixture was added to each well and incubated for two hours at 37° C.

Following incubation, the E. coli bioparticles were aspirated by vacuum aspiration, and 100 ul of trypan blue/citrate-balanced salt solution (Molecular Probes) was added to each well. After approximately 1 minute, the trypan blue was removed by vacuum aspiration and the fluorescent product was measured using a Cytofluor 4000 fluorescence microplate reader (Wavelengths: excitation—485, emission—530).

### 3.0 Material

The materials were as follows:

### Diet A—Control

Diet B—Porcine serum (PP)
Diet C—Bovine plasma protein (BP)
Diet D—Nalco-treated plasma light phase (BL)
Diet E—Nalco-treated plasma heavy phase (BH)
The dietary treatments for Experiment II were as follows:

### 1. Control

2. Ig concentrate, 2.5%
3. Ig concentrate, 0.5%
4. Bovine serum, 5%
5. Bovine serum, 1%
6. Heavy phase, 0.5%

### 7. Activated HP, 0.5%

8. Activated, de-ashed HP, 0.1%

### 3.1 Storage, and Handling of Study Material

The test diets were stored at 4° C. in their original ziploc bags. Safety glasses, gloves, and a lab coat were worn while handling.

### 3.2 Application of Study Material

Feeding dishes were filled twice a day and animals were allowed to feed ad lib for seven days.

### Results and Discussion

According to the invention we found that plasma of either bovine or porcine species origin resulted in less TNF-α production by both stimulated and unstimulated peritoneal macrophages. In addition, the administration of both the heavy and the light phase of plasma treated with 5% silicon dioxide resulted in reduced TNF-α production albeit at different concentrations. The fractions were not evaluated at equal concentrations, however. The change in TNF-α that accompanied macrophage stimulation was greater when animals were fed a plasma fraction, irrespective of source or concentration. This observation indicates that the immunological responsiveness of the macrophage is enhanced with the addition of plasma and/or it's components to the diet of young mice.

In the second experiment, we confirmed the suppressive effect of plasma fractions on TNF-α production by unstimulated peritoneal macrophages. The level of supplementation and the fraction did alter the effect however. The Nalco precipitate reduced TNF-α production in unstimulated cells at both 0.5 and. 1%. The immunoglobulin rich fraction suppressed TNF-α production at 0.5% but not at 2.5%. The addition of serum suppressed TNF-α production at 5% but not at 1.0%.

The experimental conditions in Exp. II differed from the previous experiment. The mice in this study were all challenged with endotoxin on d 1 in an attempt to prime the immune system in all animals. Previous reports have found that priming macrophages will reduce immunological responsiveness upon subsequent challenge. The results of the first experiment would seem to confirm this observation. Isolated macrophages from animals fed the control diet produced higher levels of TNF-α in the unstimulated state and therefore produced less TNF-α when stimulated with LPS than animals fed diets supplemented with plasma and/or fractions. The levels of TNF-α were markedly different in the control animals from the two experiments. TNF-α production was 15 fold higher in the first experiment than in the second experiment. Nonetheless, while immune system activation was lower in both experiments, immunological responsiveness was greater in mice fed a diet supplemented with a plasma fraction. Both TNF-α and IL-10 concentrations increased markedly with exposure of macrophages to LPS.

Plasma is rich in biologically active proteins, peptides, cytokines, and other immunomodulatory substances. The fractions of plasma administered in these experiments differed in composition and dietary inclusion rate. The effect of these fractions on TNF-α production was consistent in the two experiments. Animals fed plasma and/or fractions thereof produced less TNF-α in an unstimulated state and therefore responded with increased TNF-α production upon stimulation with endotoxin. The results of these two experiments are consistent with the concept that both the immunoglobulin-rich fractions and the silicon dioxide fractions reduce the stimulation of the immune system. The oral administration of plasma proteins or its fractions is a novel means of reducing TNF-α production and levels.

TABLE 11
The effects of bovine and porcine plasma protein administration on
immune response measures in mice.

| | TNF-α, pg/ml | | | Respiratory Burst | |
|---|---|---|---|---|---|
| | Unstimu- | Stimu- | TNF-α | Unstimu- | Stimu- |
| Treatment | lated | lated | change | lated | lated |
| Control | 1540^{a} | 1867^{a} | 322^{a} | 17.4^{a} | 23.9^{a} |
| Porcine plasma | 70^{b} | 1156^{b} | 1085^{b} | 12.2^{b} | 13.6^{b} |
| Bovine plasma | 28^{b} | 1135^{b} | 1107^{b} | 10.1^{b} | 11.1^{b} |
| Bovine plasma | 136^{b} | 1260^{b} | 1101^{b} | 10.6^{b} | 13.7^{b} |
| (Heavy phase) | | | | | |
| Bovine plasma | 34^{b} | 1135^{b} | 1124^{b} | 9.3^{b} | 11.2^{b} |
| (Light phase) | | | | | |

TABLE 12
Mean Phagocytosis Results for Peritoneal Macrophages

| | Animal | Mean | |
|---|---|---|---|
| Diet | No. | Result | Se |
| Control | 1-12 | 298 | 47.6 |
| PP | 13-24 | 264 | 46.2 |
| BP | 25-36 | 311 | 52.1 |
| BL | 37-48 | 360 | 66.5 |
| BH | 49-60 | 375 | 63.9 |

TABLE 13
TNF-α production in cultured peritoneal macrophages
from mice fed plasma protein components

| | TNF-α production, pg/ml | | |
|---|---|---|---|
| Treatment | Unstimulated | Stimulated | Change |
| Control | 128^{a} | 296^{a} | 169^{a} |
| Ig concentrate, 2.5% | 107^{ab} | 308^{a} | 201^{ab} |
| Ig concentrate, .5% | 20^{b} | 325^{a} | 306^{b} |
| Bovine serum, 5% | 5^{b} | 371^{a} | 366^{b} |
| Bovine serum, 1% | 130^{a} | 306^{a} | 176^{a} |
| Heavy phase, .5% | 48^{ab} | 271^{a} | 223^{ab} |
| Activated HP, .5% | 30^{ab} | 303^{a} | 272^{ab} |
| Activated, de-ashed | 11^{b} | 352^{a} | 341^{b} |
| HP, .1% | | | |

TABLE 14
IL-10 production in cultured peritoneal macrophages
from mice fed plasma protein components

| | IL-10 production, pg/ml | | |
|---|---|---|---|
| Treatment | Unstimulated | Stimulated | Change |
| Control | 80^{a} | 237^{a} | 156^{a} |
| Ig concentrate, 2.5% | 92^{a} | 366^{a} | 274^{a} |
| Ig concentrate, .5% | 45^{a} | 374^{a} | 329^{ab} |
| Bovine serum, 5% | 22^{a} | 369^{a} | 347^{b} |
| Bovine serum, 1% | 116^{a} | 354^{a} | 238^{ab} |
| Heavy phase, .5% | 64^{a} | 348^{a} | 284^{ab} |
| Activated HP, .5% | 54^{ab} | 394^{a} | 339^{b} |
| Activated, de-ashed | 32^{b} | 412^{a} | 381^{b} |
| HP, .1% | | | |

### Reference List

1. Eibl M M, Wolf H M, Furnkranz H, Rosenkranz A. Prevention of Necrotizing Enterocolotis in low-birth-weight infants by IgA-IgG feeding. The New England Journal of Medicine 1988;319(1):1-7.
2. Wolf H M, Eibl M M. The anti-inflammatory effect of an oral immunoglobulin (IgA-IgG) preparation and its possible relevance for the prevention of necrotizing enterocolitis. Acta Paediatr Suppl 1994;396:37-40.
3. Wolf H M, Hauber I, Gulle H, Samstag A, Fischer M B, Ahmad R U, Eibl M M. Anti-inflammatory properties of human serum IgA: induction of IL-1 receptor antagonist and Fc aR (CD89)-mediated down-regulation of tumour necrosis factor-alpha (TNF--a) and IL-6 in human monocytes. Clin. Exp. Immunol. 1996;105:537-43.
4. Caldarini d B M, Schiffrin E J, Ogawa d F, Caccamo D V, Ledesma d P M, Celener D, Bustos-Fernandez L. Prevention of carrageenan-induced ulcerative colitis in the guinea pig by serum of bovine colostrum. Medicina. (B.Aires.) 1987;47(3):273-7.
5. Donnet-Hughes A, Duc N, Serrant P, Vidal K, Schiffrin E J. Bioactive molecules in milk and their role in health and disease: the role of transforming growth factor-beta. Immunol. Cell Biol. 2000.February; 78.(1.):74.-9. 78(1):74-9.
6. Donnet-Hughes A, Schiffrin E J, Huggett A C. Expression of MHC antigens by intestinal epithelial cells. Effect of transforming growth factor-beta 2 (TGF-beta 2). Clin Exp. Immunol. 1995 Feb;99(2):240-4.
7. Zijlstra R T, McCracken B A, Odle J, Donovan S M, Gelberg H B, Petschow B W, Zuckermann F A, Gaskins H R. Malnutrition modifies pig small intestinal inflammatory responses to rotavirus. J Nutr 1999 April; 129(4):838-43.
8. Yeh S S, Schuster M W. Geriatric cachexia: the role of cytokines Am. J Clin Nutr 1999 August; 70(2):183-97.
9. Rozenfeld R A, Huang W, Hsueh W. Effects of antibiotics and germ-free environment on endotoxin (LPS)-induced injury and on intestinal group IIphospholipase A2 (PLA2-II) activity [Abstract]. In: FASEB Journal 1999;643.5

1. A method of treating chronic immune stimulation in a human, comprising orally administering to said human an effective treatment amount of a non-specific immunoglobulin concentrate derived from pig, bovine, ovine, poultry, equine or goat plasma so that serum IgG is lowered in the human, wherein the immunoglobulin concentrate contains approximately 35-50% IgG and wherein the human is afflicted with a condition characterized by chronic immune stimulation.

2. A method of treating Crohn's disease in a human, comprising orally administering to said human an effective treatment amount of a non-specific immunoglobulin concentrate derived from pig, bovine, ovine, poultry, equine or goat plasma so that serum IgG is lowered in the human, wherein the immunoglobulin concentrate contains approximately 35-50% IgG and wherein the human is afflicted with Crohn's disease.

3. A method of treating rheumatoid arthritis in a human, comprising orally administering to said human an effective treatment amount of a non-specific immunoglobulin concentrate derived from pig, bovine, ovine, poultry, equine or goat plasma so that serum IgG is lowered in the human, wherein the immunoglobulin concentrate contains approximately 35-50% IgG and wherein the human is afflicted with rheumatoid arthritis.

4. A method of treating Human Immunodeficiency Virus (HIV) in a human, comprising orally administering to said human an effective treatment amount of a non-specific immunoglobulin concentrate derived from pig, bovine, ovine, poultry, equine or goat plasma so that serum IgG is lowered in the human, wherein the immunoglobulin concentrate contains approximately 35-50% IgG and wherein the human is afflicted with Human Immunodeficiency Virus (HIV).
